(19) United States
(12) Patent Application Publication
(10) Pub. No.: US 2008/0019972 A1
(43) Pub. Date: Jan. 24, 2008

(54) Method for Amplifying Therapeutic Vaccine Activity

(75) Inventor: Jean-Marie Andrieu, Paris (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: BIOVAXIM LIMITED, LONDON (GB)

(21) Appl. No.: 10/593,838

(22) PCT Filed: Mar. 25, 2005
(86) PCT No.: PCT/FR05/00729

§ 371 Date: Oct. 31, 2006

(30) Foreign Application Priority Data
Mar. 26, 2004 (FR) 04/03178

(51) Int. Cl. A61K 39/395

(52) U.S. Cl. 424/144.1

(57) ABSTRACT

A method of amplifying activity of therapeutic vaccines including administering an effective amount of a composition including a compound that depletes or inhibits B lymphocytes to a patient sufficiently to amplify an immune reaction of T cytotoxic lymphocytes. The composition may be administered to a patient to augment the specific T cell response to a vaccine, in particulars a therapeutic vaccine, including at least one inactivated human immunodeficiency virus (HIV). In particular, the depleting compound may be an antibody, advantageously monoclonal, directed against transmembrane antigen CD20 of pre-B or mature B lymphocytes.

### RELATED APPLICATION

This is a §371 of International Application No. PCT/FR2005/000729, with an international filing date of Mar. 25, 2005 (WO 2005/092373 A1, published Oct. 6, 2005), which is based on French Patent Application No. 04/03178, filed Mar. 26, 2004.

### TECHNICAL FIELD

This invention relates to immunology, more specifically in that of active specific immunotherapy, also called therapeutic vaccine.

It concerns the use of one or several compounds that deplete the B lymphocytes of the immune system. The compounds are intended to be administered to a patient at the moment of a vaccination, in particular a therapeutic vaccination, against a tumor and/or a chronic viral, parasitic or intracellular germ infection in order to amplify and/or prolong the cytotoxic activity of the T lymphocytes against tumor cells or cells infected with a virus, a parasite or intracellular germs.

### BACKGROUND

The immune system is charged with destroying (eliminating) or preventing the proliferation (maintaining a very low concentration in the organism) of microbes, viruses and parasites that have penetrated into it. The immune system is principally formed by secondary lymphoid organs of the thymus, osseous marrow and a network of cells placed in the vicinity of cutaneous and mucous integuments. The principal secondary lymphoid organs are the external ganglions situated in the neck, armpits, groin, and internal ganglions situated at the thorax and abdomen. Peyer's patches situated along the digestive tract are also very important formations of the immune system as well as the spleen.

The immune system is formed of several types of cells that each have particular specificities. The dendritic cells stemming from monocytes of the blood are charged with ingesting minimal fractions of pathogens (viruses, bacteria, parasites) in general in the region where they have penetrated the organism, then cutting into pieces to present them at their surface in the form of peptides (protein fragments) called antigens. At the same time, the dendritic cells migrate toward the different components of the immune system.

The most numerous immune cells are lymphocytes. They circulate in the blood, but the majority of lymphocytes are found in the marrow and the lymphoid organs. In these organs, they are very capable of recognizing antigens present at the surface of the dendritic cells.

There are several types of lymphocytes:
The B lymphocytes multiply and differentiate in contact with a dendritic cell presenting the antigen. These B lymphocytes differentiate in the course of their multiplication. They then take the name of plasmocytes and reside primarily in the marrow. They produce specific antibodies for the antigenic peptides that were presented to the B lymphocytes by the dendritic cells (or other cellular types capable of presenting antigens). The antibodies are proteins circulating in the vessels and extracellular spaces. They are capable of fixing themselves onto the antigen in question, thus preparing the destruction of the cells carrying the antigen. These antibodies are also capable of neutralizing the biological activity of microorganisms circulating in the blood or the extracellular environments and preparing their destruction.
The T CD8 lymphocytes are primarily cytotoxic lymphocytes. After multiplication and maturation in the immune system and particularly in lymphoid organs, upon contact with the dendritic cell carrying antigen, these T CD8 lymphocytes acquire the capacity to destroy all the cells carrying the antigen with which they come in contact.
A third category of lymphocytes, the T CD4 lymphocytes, is absolutely indispensable for the proliferation and maturation of the B lymphocytes (that will produce antibodies) and of the T CD8 lymphocytes (that will produce cytotoxic cells). These T CD4 lymphocytes are also specific for each type of antigen. After having entered into contact with the dendritic cell in the lymphoid organs they have the particular capacity to favor the multiplication and maturation of CD8 lymphocytes (thus, in particular T CD4 lymphocytes are concerned, helpers of type 1 (TH1)), or favoring the multiplication and maturation of B lymphocytes specific for the one antigen or the other (T CD4 TH2 lymphocytes are then concerned).

The immune system is thus particularly efficient in controlling or, better yet, eliminating from the organism cells infected by different types of microorganisms such as viruses, microbes, parasites. Certain types of pathogens necessitate chiefly or exclusively turning on B lymphocytes at the origin of the production of antibodies. It is said that they put the humoral immunity in play. Conversely, certain viruses, parasites or microbes situated inside cells preferentially necessitate turning on TCD8 lymphocytes capable of destroying these infected cells. These CD8 lymphocytes are responsible for what is called “cellular immunity.”

A therapeutic vaccination consists of the administration of antigens specific for tumors or microbes, viruses or parasites. These specific antigens can be administered to the patient in the form of infected or tumor cells, previously inactivated (by a physical or chemical means). They can also be administered in the form of proteins or peptides, but also DNA or RNA specific for the proteins or peptides in question. These DNAs or RNAs can be free themselves or introduced in the viral or non-viral DNA or RNA vectors. Whatever the type and the form of the preparation administered, most frequently subcutaneously (intramuscular and intravenous paths can also be used as well as the per os path), the final objective is that this preparation is finally transformed into peptides presented to the surface of the dendritic cells (or of other cell types capable of presenting the antigen).

The success of a vaccination, whatever the form of the antigenic preparation, most frequently necessitates the concomitant use of adjuvants of mineral or chemical origin or of biological compounds of natural origin.

The antigenic preparation (tumor cell or inactivated infected cell, proteins, peptide, DNA, RNA) can also be administered in vitro to dendritic cells prepared ex vivo from blood cells by virtue of cytokines. These dendritic cells are modified by the action of the cytokines to express specific antigens (peptides) on their surface. Once they have been put in contact in an appropriate fashion with the antigenic preparation, these dendritic cells charged ex vivo in antigens are administered to the patient, most frequently by subcutaneous injection. These cells migrate toward the lymphoid organs and bring about the proliferation and differentiation of the T8 lymphocytes cytotoxic to tumor cells or infected cells (that carry the antigens in question) and/or to the B lymphocytes that, transforming themselves into plasmocytes producing antibodies against the antigenic peptides carried by the tumor cells or the infected cells, can allow the lysis of these cells to be facilitated.

It can be expected from the principle of therapeutic vaccination that it favors the control of or even eradication of chronic infections or tumors. The clinical results of these therapeutic vaccines against different types of cancers or against chronic infections have been mediocre.

### SUMMARY

This invention relates to a method of amplifying activity of therapeutic vaccines including administering an effective amount of a composition including a compound that depletes or inhibits B lymphocytes to a patient sufficient to amplify an immune reaction of T cytotoxic lymphocytes.

### BRIEF DESCRIPTION OF THE DRAWING

FIG. 1 is a graph showing the number of copies of RNA per milliliter of plasma as a function of time after vaccination.

### DETAILED DESCRIPTION

That which was previously stated explains the importance of the cytotoxic T lymphocytes as well as that of the B lymphocytes in the immune reaction and, in particular, in the potential success of therapeutic vaccines. According to the current state of knowledge, the presence and activity of B lymphocytes appears to be essential to obtain a satisfactory immune reaction.

We determined in a surprising and unexpected manner that a compound that depletes or inhibits naive B lymphocytes of the lymphoid organs is capable of amplifying the immune reaction of the cellular type.

In fact, we observed that administration of a compound that depletes naïve B lymphocyte allowed the augmentation of the T cell response specific for a therapeutic vaccine formed by an immunodeficiency virus of the monkey (SIV) inactivated by a chemical method and from an adjuvant (incomplete Freund's adjuvant) and administered subcutaneously to monkeys infected by the monkey immunodeficiency virus.

This surprising result goes against the general knowledge concerning the immune reaction such as was briefly discussed above. In fact, it appears that the depletion of B lymphocytes when it is associated with a vaccination for stimulating cellular immunity not only seems to not delete, but seems to favor the effectiveness of the reaction of cellular immunity.

Therefore, we provide a compound that depletes or inhibits B lymphocytes, particularly the naive B lymphocytes, in the preparation of a composition for being administered to patients in order to amplify an immune reaction of the T cytotoxic lymphocytes.

This composition can be advantageously administered to a patient to amplify an immune reaction of the T cytotoxic lymphocytes when it is excited by a vaccination, preferably a therapeutic vaccination, against a tumor and/or against a chronic viral, parasitic or intracellular germ infection.

The composition can be particularly used in the treatment of tumor diseases with the exception of diseases of the hematopoietic and immune system such as leukemias and lymphomas, particularly B lymphoma.

The composition that depletes or inactivates the B lymphocytes can be any compound whose administration brings about a depletion of the B lymphocytes or at least an inactivation of the B lymphocytes, that is, a compound whose administration has as a consequence a diminution or even a complete transitory halt of the activity of B lymphocytes.

The compound can be, e.g., a monoclonal or polyclonal antibody, in particular, an antibody directed against B lymphocytes. In particular, the compound can be an antibody directed against transmembrane antigen CD20 of pre-B or mature B lymphocytes. The antibody is preferably a monoclonal antibody directed against transmembrane antigen CD20 of pre-B or mature B lymphocytes.

The antibody can be a natural antibody or obtained by genetic engineering. The antibody can be of human origin or of any other mammal such as e.g., murine or also produced by genetic engineering such as, e.g., in microorganisms or even by chemical synthesis.

The antibody can be humanized or not humanized. It can be a chimeric or recombined antibody. In particular, the antibody can be the monoclonal antibody sold under the name of RITUXIMAB®. It is then a murine/human chimeric antibody obtained by genetic engineering. It is a glycosylated immunoglobulin associating on the one hand the constant regions of a human IgG1 and on the other hand the variable regions of light and heavy chains of murine origin. The antibody may be produced by a culture of mammalian cells (Chinese hamster ovaries) and purified by affinity and ion exchange chromatography comprising specific processes of viral inactivation and elimination.

The antibody, particularly its fragment Fab, binds specifically to a CD20 transmembrane antigen of B lymphocytes. This antigen is not internalized during binding to the antibody and it is not freed from the cellular surface. The CD 20 does not circulate in a free form in the plasma and therefore does not compete for binding to the antibody.

Once bound to antigen CD 20 of the B lymphocytes, the complex formed between the antibody, or its Fab fragment, and antigen CD 20generates functions of immune effector that bring about the lysis of the lymphocytes via fragment Fc. The possible mechanisms of cellular lysis are cytotoxicity dependent on the compliment (CDC) that brings about the intervention of the binding of the fragment Cl1, and cellular cytotoxicity dependent on the antibodies (ADCC) passing via one or several of the gamma Fc receptors of the surface of granulocytes, macrophages and NK cells.

Therefore, the compound that depletes or inactivates B lymphocytes can be a Fab fragment of an antibody directed against transmembrane antigen CD 20 of pre-B or mature B lymphocytes.

The composition in the preparation of which the compound that depletes or inhibits B lymphocytes is used can be administered by any known means prior to, concomitant with or subsequent to a vaccination, in particular a therapeutic vaccination against a tumor and/or against a chronic viral, parasitic or intracellular germ infection. Administration of the composition can be realized by any known means. The following can be cited by way of example: injection, in particular subcutaneous or intravenous or intramuscular injection or also oral administration. Administration is preferably performed by an intravenous injection.

The composition can comprise any known support biologically compatible for administration to a patient. The following can be cited by way of example: sterile demineralized water, physiological serum or also a solution for perfusion.

A particularly preferred use is the use of a compound that depletes or inhibits B lymphocytes in the preparation of a composition to be administered to a patient to augment the T cell response specific for a therapeutic vaccine, comprising at least one inactivated human immunodeficiency virus (HIV). In particular, the depleting compound is an antibody, advantageously monoclonal, directed against transmembrane antigen CD20 of pre-B or mature B lymphocytes.

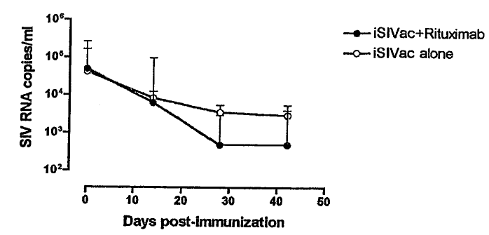

FIG. 1 shows the result obtained by a therapeutic vaccination performed on monkeys infected by monkey immunodeficiency virus (SIV) and treated or not treated according to aspects of the invention with a monoclonal antibody directed against transmembrane antigen CD20 of B lymphocytes (RITUXIMAB®). The curves represent the number of copies of RNA of the SIV virus per milliliter of plasma of monkeys infected by the SIV that received a therapeutic vaccine one year after the infection composed of inactivated SIV virus and of adjuvant, and treated or not treated with RITUXIMAB®, as a function of the number of days after the therapeutic vaccination.

FIG. 1 shows the results obtained with:
∘: An inactivated SIV vaccine+adjuvant;
● An inactivated SIV vaccine+adjuvant preceded (d−3) and followed (d+4 and d+11) by an administration of RITUXIMAB®.

Other characteristics will be apparent from the following representative example as well as from the figure without constituting any limitation on the scope of the appended claims.

### EXAMPLE

### Measure of the Effect of a Compound that Depletes B Lymphocytes on the Response of T Cells After Therapeutic Vaccination Against a Virus

Materials and Methods:
The research project was approved by the committee for animal studies of the Institute of Tropical Medicine of Guangzhou, China.
Preparation of the inactivated virus: the virus SIVmac251 was inactivated by treatment with aldrithiol-2 (AT-2) as previously described (W. Lu, et al., J. Virol. 75: 8949-8956, 2001). The inactivated SIV-AT-2 virus was concentrated by ultracentrifugation in order to obtain a final concentration of 2.10¹⁰ viral particles/ml, and was then congealed at −80° C. for preservation until its use.
Animals: 8 healthy adult macaques, rhesus “colony-bred” from the Shunde Experimental Animal Centered (Guangdong, China). The animals had been infected with the SIVmac251 virus as previously described (W. Lu et al., Nat. Med. 9: 27-32, 2003) one year prior to the therapeutic vaccination.
Preparation of the therapeutic vaccine: The inactivated SIV-AT-2 virus was thawed to ambient temperature. 10¹⁰ viral particles (0.5 mm) were then mixed to 0.5 ml incomplete Freund's adjuvant (Sigma-Aldrich Chimie Sarl, Saint Quentin Fallavier, France) in order to yield 1 ml of inactivated SIV-AT-2 vaccine. This mixture was used to immunize the animals.
Vaccination: the 8 animals received 1 subcutaneous injection of 0.25 ml at the root of 4 members (or a total of 1 ml) of inactivated SIV-AT-2 vaccine. 4 of them received RITUXIMAB® intravenously at the rate of 10 mg/kg, 3 days prior to the therapeutic vaccination then 4 days and 11 days after this therapeutic vaccination.
Virological and immunological measures: The measure of the viral charge and of the virospecific cytolysis were performed regularly every 2 weeks as previously described (W. Lu et al., J. Virol., 75: 8949-8956, 2001; W. Lu et al., Nat. Med.: 1081-1885, 1999) without modification. The response of the CD4⁺ Th1 cells and of the memory CD8⁺ T cells was measured by the spot test of the secretion of γ-interferon with the aid of the ELISPOT kit of R&D Systems Europe (Lille, France) according to the recommendations of the supplier.
Statistical analyses: The tests of Mann-Whitney or of Wilconon were used to compare the data before and after immunization.

Results:

Measure of the effect of the compound depleting B lymphocytes:

4 weeks after the therapeutic vaccination the quantity of RNA of the monkey immunodeficiency virus (SIV) contained in the plasma of the monkeys had diminished by 100 times in the animals vaccinated and treated with RITUXIMAB® and by 10 times in the animals vaccinated but not treated with RITUXIMAB®, FIG. 1 shows these results.

### CONCLUSION

The temporary depletion or inhibition of naïve B lymphocytes is a powerful tool in the promotion of the specific antigen cytotoxic response of the T cells in the course of immunization against viruses or tumors.

1-13. (canceled)

14. A method of amplifying activity of therapeutic vaccines comprising administering an effective amount of a composition comprising a compound that depletes or inhibitis B lymphocytes to a patient sufficient to amplify an immune reaction of T cytotoxic lymphocytes.

15. The method according to claim 14, wherein the B lymphocytes are naïve B lymphocytes.

16. The method according to claim 14, wherein the immune reaction of the T cytotoxic lymphocytes is excited by a vaccination.

17. The method according to claim 16, wherein the vaccination is a vaccination against a tumor and/or against a chronic viral, parasitic or intracellular germ infection.

18. The method according to claim 16, wherein the vaccination is a therapeutic vaccination.

19. The method according to claim 14, wherein the compound that depletes or inactivates the B lymphocytes is a monoclonal or polyclonal antibody or a Fab fragment of an antibody.

20. The method according to claim 19, wherein the antibody or the Fab fragment of the antibody is directed against transmembrane antigen CD20 of the pre-B or mature B lymphocytes.

21. The method according to claim 19, wherein the antibody is a monoclonal antibody.

22. The method according to claim 19, wherein the antibody is a murine/human chimeric antibody obtained by genetic engineering.

23. The method according to claim 14, wherein the composition is administered prior to and/or concomitant with and/or subsequent to a vaccination against a tumor and/or against a chronic viral, parasitic or intracellular germ infection.

24. The method according to claim 23, wherein the vaccination is a therapeutic vaccination.

25. The method according to claim 14, wherein the composition is administered to augment specific T cell response to a therapeutic vaccine comprising an inactivated human immunodeficiency virus (HIV).

26. The method according to claim 25, wherein the depleting compound is a monoclonal antibody directed against transmembrane antigen CD20 of pre-B or mature B lymphocytes.
